(19) United States
(12) Patent Application Publication
(10) Pub. No.: US 2025/0169939 A1
(43) Pub. Date: May 29, 2025

(54) POLYMER SCAFFOLD FOR PROSTHESIS WITH ADJUSTABLE INTERNAL RIGIDITY AND ELASTICITY, AND METHOD OF MANUFACTURING THE SAME

(71) Applicant: ARC KOREA CO., LTD., Seongnam-si, Gyeonggi-do (KR)

(72) Inventor: Jung-Wan SON, Seongnam-si, Gyeonggi-do (KR)

(21) Appl. No.: 18/841,350

(22) PCT Filed: Jan. 13, 2023
(86) PCT No.: PCT/KR2023/000618

§ 371 Date: Aug. 23, 2024

(30) Foreign Application Priority Data
Mar. 29, 2022 (KR) 10-2022-0038636

(51) Int. Cl. A61F 2/08; A61L 27/44; A61L 27/56; B33Y 80/00

(52) U.S. Cl. CPC A61F 2/08; A61L 27/44; A61L 27/56; B33Y 80/00; A61F 2210/0004; A61F 2210/0076; A61F 2240/001; A61F 2250/0015; A61F 2250/0023; A61F 2250/0036; A61L 2430/10

(57) ABSTRACT

The present invention is directed to providing a polymer scaffold for a prosthesis that may have a size and supporting force that may accurately correspond to a certain affected area, may also contain a maximum amount of a therapeutic drug, may be immediately applied in a customized manner to an affected area that has not been prepared in advance, and may provide a stable supporting force of the affected area throughout the treatment period, and a method of manufacturing the same. The polymer scaffold for a prosthesis according to the present invention and the method of manufacturing the same have an effect of allowing the internal rigidity and elasticity to be adjusted so that they can be provided in a customized manner to patients.

### TECHNICAL FIELD

The present invention relates to a polymer scaffold for a prosthesis made of a biodegradable polymer material for stabilization and regeneration of an affected area and implanted into the affected area to support the affected area, and a method of manufacturing the same.

### BACKGROUND ART

In the past, surgery was typically completed by wrapping the ligaments and tissues with sutures even when a space was created due to tissue damage or disconnection at the surgical site, such as the inside of muscle, joints, or the like.

For example, in the case of the rotator cuff repair shown in FIGS. 1 and 2, surgery is not performed while directly observing the inside of the joint, but is performed by incising a part of the joint and inserting an arthroscope into the joint while looking at the monitor screen.

As a result, the distance between the end of the surgical instrument and the hand is long, and the surgical instrument must be controlled indirectly through a monitor. Therefore, even when the surgeon is extremely skilled, a long surgery lasting more than an hour must be performed under general anesthesia.

Also, in order for the regenerative tissue at the surgical site to recover and the sutured state to be established after surgery, consistent rehabilitation movements must be performed within a set period of time.

However, when the muscles have not recovered because there is an empty space at the surgical site, the rehabilitation movements cause considerable pain, and thus a rehabilitation process cannot help to be a series of tremendous pain.

To solve this problem, as shown in U.S. Pat. No. 9,770,337 B2, technology has been developed in the art to protect a surgical site by inserting a tube-shaped instrument into a surgical site to fill a liquid into the tube through a special nozzle during the surgical procedure and to ease the rehabilitation process by minimizing the movement of the surgical site during the rehabilitation process to significantly reduce pain.

However, in the related art, because the leakage of the liquid filled in the tube must be prevented, the nozzle used to inject the liquid into the tube must be installed with precision instrument having a special structure at the ends thereof so that no nozzle insertion holes are left in the tube after the nozzle is removed from the tube.

Also, since the tube must be prevented from melting inside the body in order to prevent the leakage of the liquid, a separate surgery to remove the tube is required later, which delays the patient's recovery and significantly increases costs.

In addition, the related art discloses that the ligament tissue repair function may be performed by attaching a biodegradable polymer (e.g., collagen, etc.) to the ligament during a ligament restoration procedure, but has problems in that the strength of the biodegradable hydrogel support has weak when used alone, and it is difficult to fix collagen and the like on the target body tissue surface, and also has a problem in that the collagen and the like easily become loose in the body after the procedure.

Therefore, there is a need for a technology that can reduce costs and eliminate stress by eliminating the need for subsequent removal surgery while maintaining the effect of stabilizing the surgical site and reducing pain, as in the related art.

In particular, although the drug delivery speed needs to be adjusted because the speed of recovery or tissue regeneration differs depending on the type of affected area or the age of a patient, it is still difficult to find a technology for biodegradable drug delivery and tissue regeneration scaffolds with a means capable of controlling the drug delivery speed.

Further, although the type and size of the affected area may both be different depending on the type and severity of the injury, and the recovery speed and tissue regeneration speed may both be different depending on the age, body type, and physique of a patient even when patients have the same type of injury having similar severity, technology for implantable prostheses capable of being provided in a customized manner to patients is still inadequate.

### RELATED-ART DOCUMENT

U.S. Pat. No. 9,770,337 (Registration date: Sep. 26, 2017)

### DISCLOSURE

### Technical Problem

Therefore, the present invention is directed to providing a polymer scaffold for a prosthesis that may have a size and supporting force that may accurately correspond to a certain affected area, may also contain a maximum amount of a therapeutic drug, may be immediately applied in a customized manner to an affected area that has not been prepared in advance, and may provide a stable supporting force of the affected area throughout the treatment period, and a method of manufacturing the same.

### Technical Solution

To solve these problems, a polymer scaffold for a prosthesis according to the present invention includes an inner sheet formed into a certain size and shape and made of a biodegradable synthetic polymer material, and an outer shell made of a biodegradable natural polymer material in the form of a sealed pouch surrounding the inner sheet, wherein the inner sheet is formed by cutting a portion of a synthetic polymer matrix, which is made of the biodegradable synthetic polymer material and has a larger area and thickness than the inner sheet, into a predetermined shape and size, so that the synthetic polymer matrix has a network-like structure in which fine nanofibers are formed by amorphous stacking, which makes it possible to control the diameter and the stacking density of the fine nanofibers during a stacking process of the fine nanofibers, so that a material of the inner sheet can be determined depending on the diameter and the stacking density of the fine nanofibers constituting the inner sheet, and the overall shape and elasticity characteristics can be determined depending on the shape, size, and thickness of the inner sheet, or the inner sheet is formed by 3D printing, and the inner sheet is formed with bends or folds at regular intervals along the length or width direction in a shape corresponding to a predetermined size and shape of the affected area to form an uneven shape, or is formed with bends or folds partially or entirely, or is formed in a flat or gently curved shape or a certain geometric shape, and the inner sheet is formed of one layer of one sheet-shaped member or formed by overlapping two or more layers of one sheet-shaped member.

Also, a material of the synthetic polymer matrix may preferably include a component composed of any one or a combination of two or more selected from poly (L-lactic acid) (PLA), poly (glycolic acid) (PLGA), poly (ε-caprolactone) (PCL), and poly (L-lactide-co-ε-carprolactone (PLCL).

In addition, the natural polymer material may preferably include a component composed of any one or a combination of two or more selected from collagen type 1, sodium hyaluronate (HA), and chondroitin sulfate.

Meanwhile, a method of manufacturing a polymer scaffold for a prosthesis according to the present invention includes manufacturing a mold in a shape corresponding to a predetermined shape of an affected area, manufacturing an inner sheet by mixing a biodegradable synthetic polymer fuel, inserting the inner sheet into the mold, preparing a natural polymer solution by mixing a biodegradable natural polymer fuel, injecting the natural polymer solution into the mold, drying the natural polymer solution injected into the mold, and removing the mold, wherein the size and shape of the polymer scaffold can be adjusted to correspond to the affected area of the patient and treatment period by changing the size and shape of the mold according to the age of a patient, the size of the affected area, and the treatment period.

Here, the manufacturing of the synthetic polymer matrix may preferably include dispersing the synthetic polymer solution by spraying the synthetic polymer solution through a piston nozzle, wherein the synthetic polymer sprayed from the nozzle may be formed in the form of fine fiber strands while weakening the surface tension of the synthetic polymer by disposing a high-voltage electrode at the nozzle and applying a high voltage to the nozzle, and the fiber form may be produced by sequentially amorphously stacking the fiber strands on a predetermined collector plate having a certain area.

Also, the manufacturing of the inner sheet may include manufacturing the inner sheet by injecting the biodegradable synthetic polymer fuel by 3D printing to mold the biodegradable synthetic polymer fuel into a specific shape.

In addition, the manufacturing of the synthetic polymer matrix may preferably include adjusting the porosity and density of the completed synthetic polymer matrix by increasing or decreasing the viscosity of the synthetic polymer solution to reduce or increase the thickness of the fiber strands, increasing or decreasing the magnitude of the high voltage to reduce or increase the thickness of the fiber strands, and decreasing or increasing the inflow speed of the synthetic polymer solution introduced into the piston nozzle to reduce or increase the thickness of the fiber strands.

In this case, the manufacturing of the synthetic polymer matrix may preferably include further decreasing the diameter of the fiber strands spun from the piston nozzle by adding a salt to the synthetic polymer solution to increase the surface charge density of the synthetic polymer sprayed from the piston nozzle.

Meanwhile, the manufacturing of the inner sheet matrix may preferably include cutting a portion of the synthetic polymer matrix from the synthetic polymer matrix to an area and thickness that are insertable into the mold, and forming an uneven shape by forming bends or curves on the cut portion of the synthetic polymer matrix at regular intervals along the length direction in a shape corresponding to a predetermined size and shape of the affected area, or forming the cut portion into a three-dimensional shape by performing bend or curve processing on part or the entirety of the cut portion.

Meanwhile, when the biodegradable synthetic polymer fuel is injected by 3D printing to mold the biodegradable synthetic polymer fuel into a specific shape, the specific shape may be produced by forming curves, protrusions, or perforations at regular intervals, forming the inner sheet to have a gentle curve over the entire area thereof, or molding the inner sheet to have a specific geometric shape, and the inner sheet is formed of one sheet or formed by overlapping two or more sheets.

### Advantageous Effects

The polymer scaffold for a prosthesis according to the present invention and the method of manufacturing the same have an effect of allowing the internal rigidity and elasticity to be adjusted so that they can be provided in a customized manner to patients because the type and size of the affected area may both be different depending on the type and severity of the injury, and the recovery speed and tissue regeneration speed may both be different depending on the age, body type, and physique of a patient even when the patient have the same type of injury having similar severity.

### DESCRIPTION OF DRAWINGS

FIGS. 1 and 2 are diagrams showing conventional rotator cuff repair.

FIG. 3 is a perspective view of a polymer scaffold for a prosthesis according to the present invention.

FIG. 4 is a cross-sectional side view of FIG. 2.

FIG. 5 is a plan view of FIG. 2.

FIG. 6 is a conceptual diagram showing electrospinning in a method of manufacturing a polymer scaffold for a prosthesis according to the present invention.

FIG. 7 is a conceptual diagram showing some principles for electrospinning of FIG. 5.

FIG. 8 is a conceptual diagram showing mold forming in the method of manufacturing a polymer scaffold for a prosthesis according to the present invention.

FIG. 9 is a conceptual diagram showing various embodiments of the shape of the inner sheet inserted into the mold in FIG. 8.

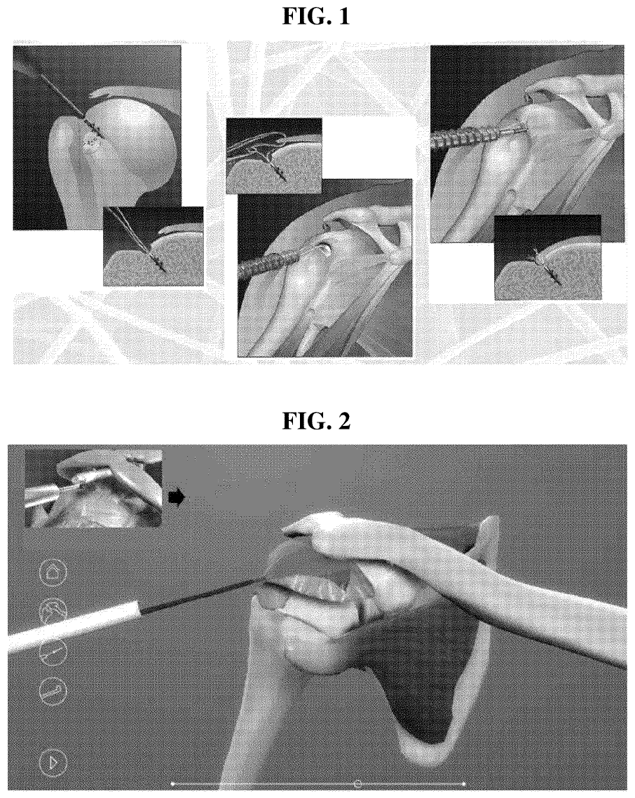

### BEST MODE

Specific structural or functional descriptions presented in embodiments of the present invention are merely illustrative for the purpose of describing the embodiments according to the concept of the present invention, and the embodiments according to the concept of the present invention may be implemented in various forms. Also, it should be understood that the present invention is not intended to be limited to the embodiments described in this specification, and includes all modifications, equivalents, and substitutions which fall within the spirit and technical scope of the present invention.

Hereinafter, the present invention will be described in detail with reference to the accompanying drawings.

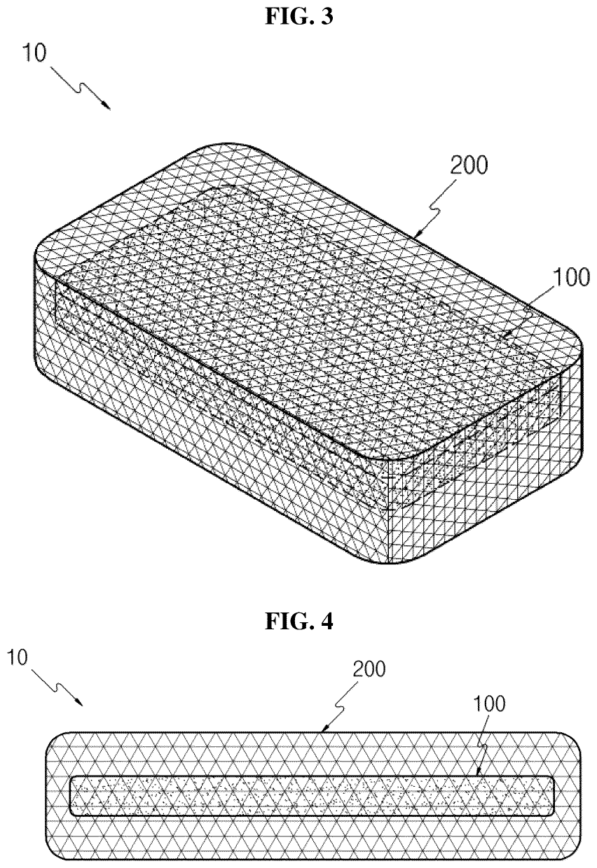

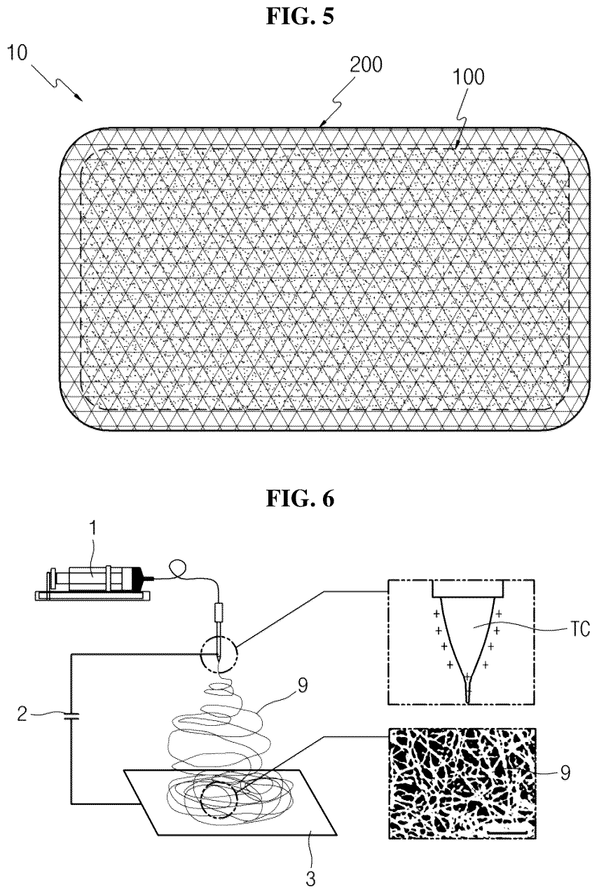

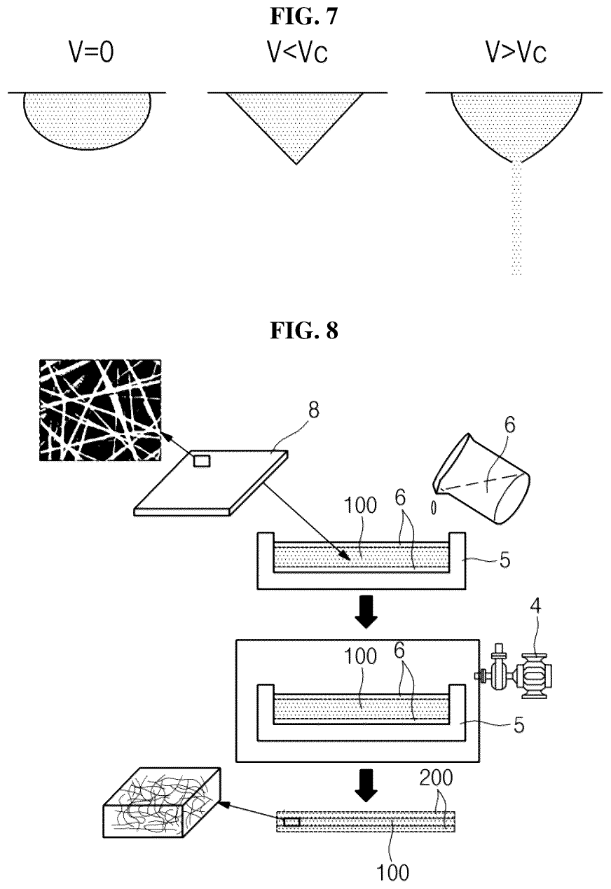

As shown in FIG. 3, a polymer scaffold 10 for a prosthesis according to the present invention includes an inner sheet 100 formed into a certain size and shape and made of a biodegradable synthetic polymer material, and an outer shell 200 in the form of a sealed pouch surrounding the inner sheet 100 and made of a biodegradable natural polymer material.

Here, there may be two embodiments for manufacturing the inner sheet 100.

### First Embodiment of Inner Sheet

The first embodiment is an embodiment in which a biodegradable synthetic polymer matrix manufactured in advance is first manufactured as shown in FIG. 8 and the inner sheet 100 is then formed by cutting a portion of the biodegradable synthetic polymer matrix into a predetermined shape and size.

In this case, the synthetic polymer matrix 8, which is provided for the manufacture of the inner sheet 100 and serves as a base of the inner sheet 100, has a network-like structure in which fine nanofibers 9 are formed by amorphous stacking, which makes it possible to control the diameter and the stacking density of the fine nanofibers 9 during a stacking process of the fine nanofibers 9, so that a material of the inner sheet 100 can be determined depending on the diameter and the stacking density of the fine nanofibers 9 constituting the inner sheet, and the overall shape and elasticity characteristics (i.e., a supporting force of the affected area) can be determined depending on the shape, size, and thickness of the inner sheet 100.

That is, since the inner sheet 100 is formed of a network structure of fine nanofibers 9, the diameter and stacking density of the fine nanofibers 9 may be controlled during a process of manufacturing the synthetic polymer matrix 8, which is a base of the inner sheet 100, through electrospinning, which will be described later. Therefore, the inner sheet 100 has the effect of enabling the manufacture of inner sheets 100 having various densities, elasticities, dissolution rates, and porosities.

More specifically, as shown in FIG. 6, the synthetic polymer matrix 8 is formed into a micro-network structure by amorphously stacking synthetic polymer fibers that are injected by applying a high voltage to a high-voltage electrode 2 connected to a piston nozzle 1, and a plurality of synthetic polymer matrices 8 having different densities and porosities may be provided by differently forming the thickness and stacking distance of the fine nanofibers 9 depending on the viscosity of the synthetic polymer solution filled in the piston nozzle 1 and the voltage magnitude of the high-voltage electrode 2. As a result, since it is possible to manufacture a plurality of synthetic polymer matrices 8 having different densities and porosities on the spot, the inner sheet 100 may be manufactured in a customized manner to fit a certain patient and affected area.

### Second Embodiment of Inner Sheet

In this embodiment, the inner sheet is manufactured on the spot by 3D printing.

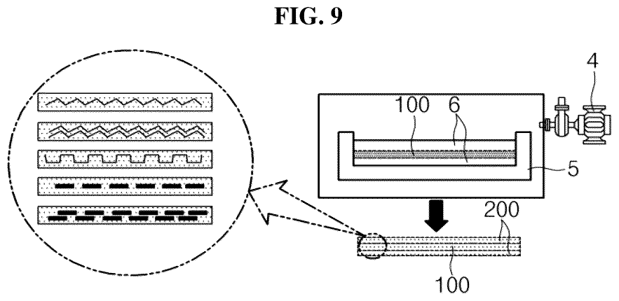

Here, when the biodegradable synthetic polymer fuel is injected by 3D printing to mold the biodegradable synthetic polymer fuel into a specific shape, the specific shape may be produced by forming curves, protrusions, or perforations at regular intervals, forming the inner sheet to have a gentle curve over the entire area thereof, or molding the inner sheet to have a specific geometric shape, as shown in the lower left of FIG. 9, and the inner sheet may be formed of one sheet or formed by overlapping two or more sheets.

Also, in the second embodiment of the polymer scaffold 10 for a prosthesis according to the present invention, since the outer shell 200 made of a natural polymer material is manufactured by pouring and drying a natural polymer solution 6 in a state in which the inner sheet 100 is inserted into the mold 5, as shown in FIGS. 3 and 4, there is no need to prepare the shape of the outer shell 200 in advance by processing the outer shell 200 to correspond to the shape of the inner sheet 100 even when the inner sheet 100 has an unique shape. That is, even when the outer shell 200 is manufactured to fit the shape and size of the affected area, the shape and size of the inner sheet 100 do not need to follow those of the outer shell 200.

As a result, in the present invention, the shape of the inner sheet 100 may be produced by forming folds or bends at regular intervals along the length or width direction in a shape corresponding to a predetermined size and shape of the affected area as shown in FIG. 9 to form an uneven shape, or may be formed with folds or bends over part or the entirety of the inner sheet 100, or may be formed in a flat or gently curved shape.

In this case, even when the entire scaffold 10 including the outer shell 200 has the same shape depending on the shape of the inner sheet 100, the elasticity characteristics, the dissolution rate of the inner sheet 100, or the amount that the outer shell (200) occupies in the total volume may be controlled. Therefore, even if the outer shell 200 is formed thickly, when the inner sheet 100 is formed in a curved shape such that the inner sheet 100 is evenly distributed in the internal space as shown in FIG. 9, the occupied volume of the outer shell 200 containing a drug is maximized, and the rigidity of the outer shell 200 is also maintained at a certain level or higher due to the even distribution of the inner sheet 100, thereby stably supporting the affected area.

For reference, in both of the two embodiments, the material of the synthetic polymer matrix may include a component composed of any one or a combination of two or more selected from poly (L-lactic acid) (PLA), poly (glycolic acid) (PLGA), poly (ε-caprolactone) (PCL), and poly (L-lactide-co-ε-carprolactone (PLCL).

Also, the natural polymer material constituting the outer shell may include a component composed of any one or a combination of two or more selected from collagen type 1, sodium hyaluronate (HA), and chondroitin sulfate.

The method of manufacturing a polymer scaffold 10 for a prosthesis according to the present invention taking advantage of these characteristics may be implemented in two forms corresponding to the two embodiments as described above.

### Manufacturing Method According to the First Embodiment

As shown in FIG. 8, the manufacturing method according to the present invention includes mixing a biodegradable synthetic polymer fuel to prepare a synthetic polymer solution and then manufacturing a synthetic polymer matrix 8 using the synthetic polymer solution, manufacturing a mold 5 in a shape corresponding to a predetermined shape of an affected area, manufacturing an inner sheet by cutting the synthetic polymer matrix 8 to an area and thickness that are insertable into the mold 5, inserting the inner sheet 100 into the mold 5, mixing a biodegradable natural polymer solution 6 to prepare a natural polymer solution 6, injecting the natural polymer solution 6 into the mold 5, drying the natural polymer solution 6 injected into the mold 5, and removing the mold 5. In this case, when the inner sheet 100 is formed in a curved shape such that the inner sheet 100 is evenly distributed in the inner space, the occupied volume of the outer shell 200 containing a drug is maximized, and the rigidity of the outer shell 200 is also maintained at a certain level or higher due to the even distribution of the inner sheet 100, thereby stably supporting the affected area.

Here, the manufacturing of the synthetic polymer matrix 8 may include dispersing the synthetic polymer solution by spraying the synthetic polymer solution through a piston nozzle 1, wherein the synthetic polymer sprayed from the nozzle 1 may be formed into fine nanofibers 9 while weakening the surface tension of the synthetic polymer by disposing a high-voltage electrode 2 at the piston nozzle 1 and applying a high voltage to the piston nozzle 1, and the fine nanofibers 9 may be formed by an electrospinning process by amorphously stacking the fiber strands on a predetermined collector plate 3 having a certain area.

Here, the porosity and density of the completed synthetic polymer matrix 8 may be adjusted by increasing or decreasing the viscosity of the synthetic polymer solution to reduce or increase the thickness of the fine nanofibers 9, increasing or decreasing the magnitude of the voltage to reduce or increase the thickness of the fine nanofibers 9, and adjusting the inflow speed of the synthetic polymer solution introduced into the piston nozzle 1 to adjust the thickness of the fine nanofibers 9.

In this case, the diameter of the fine nanofibers 9 manufactured by electrospinning is greatly affected by the spinning conditions of the synthetic polymer solution.

The first factor of the spinning conditions of synthetic polymer solutions is the voltage. High voltage may cause a larger amount of the polymer solution to be introduced into the jet, which may increase the diameter of the nanofibers. Also, in the case of polymers made of natural materials such as silk, the diameter of the nanofibers may decrease as the voltage increases. In particular, the high surface charge density induces high mobility of ions, which leads to a large electrostatic repulsion, thereby greatly increasing elongation and further decreasing the diameter of the nanofibers.

The second factor is the viscosity of the polymer solution. In electrospinning, in the case of a solution with low viscosity, the polymer accumulates in the form of droplets due to surface tension. At this time, as the concentration gradually increases, the polymer flies through the air without any collapse, and the spindle-shaped droplets are connected to each other by fine threads to form stable continuous fibers. Also, as the viscosity of the solution increases, the degree of entanglement of the polymer chains in a solvent increases, thereby preventing the collapse of the jet (cone jet, liquid jet, or initial jet). As a result, the jet takes the form of elongated fibers.

When the speed at which the polymer solution is introduced into the piston nozzle 1 is controlled to make the inflow speed faster, the size of the droplets formed at the tip of the piston nozzle 1 increases, and even when the droplets reach the collector plate 3 after spinning, the solvent is not completely evaporated. Therefore, the finally obtained layer of nanofibers 9 may include a structure in which bead-shaped fibers or multiple strands of fibers intersect each other.

Therefore, the smaller the inflow speed of the polymer solution, the smaller the diameter of the obtained nanofibers 9, and the larger the inflow speed of the polymer solution, the larger the diameter of the obtained nanofibers 9.

Also, the manufacturing of the synthetic polymer matrix 8 may include further decreasing the diameter of the strands of nanofibers 9 spun from the piston nozzle 1 by adding a small amount of salt to the synthetic polymer solution to increase the surface charge density of the synthetic polymer solution sprayed from the piston nozzle. This is because the added salt induces a higher charge density to be formed on the surface of the polymer solution to be discharged. Accordingly, this is because the mobility of the polymer solution increases further and a larger electrostatic repulsion force is generated, resulting in larger elongation.

### Manufacturing Method According to the Second Embodiment

As shown in FIG. 9, the manufacturing method according to the present invention includes manufacturing a mold 5 in a shape corresponding to a predetermined shape of an affected area, mixing a biodegradable synthetic polymer fuel to prepare a synthetic polymer solution and then manufacturing an inner sheet into a desired shape on the spot by 3D printing using the synthetic polymer solution, inserting the inner sheet 100 into the mold 5, preparing a natural polymer solution 6 by mixing a biodegradable natural polymer fuel 6, injecting the natural polymer solution 6 into the mold 5, drying the natural polymer solution 6 injected into the mold 5, and removing the mold 5. In this case, when the inner sheet 100 is formed in a curved shape such that the inner sheet 100 is evenly distributed in the internal space, the occupied volume of the outer shell 200 containing a drug is maximized, and the rigidity of the outer shell 200 is also maintained at a certain level or higher due to the even distribution of the inner sheet 100, thereby stably supporting the affected area.

The shape of the inner sheet 100 may be produced on the spot by 3D printing. As a result, the characteristics of the entire scaffold 10, such as elastic strength, yield strength, or the like, may be changed as needed by extracting different shapes of the inner sheet 100. Such elastic strength or yield strength may be adjusted to a level corresponding to the supporting force of the scaffold 10 inserted into the affected area depending on the size and type of the affected area.

That is, in the polymer scaffold 10 for a prosthesis according to the present invention, since the outer shell 200 made of a natural polymer material is manufactured by pouring and drying a polymer solution in a state in which the inner sheet 100 is inserted into the mold 5, as shown in FIG. 9, there is no need to prepare the shape of the outer shell 200 in advance by processing the outer shell 200 to correspond to the shape of the inner sheet 100 even when the inner sheet 100 has an unique shape.

As a result, in the present invention, the shape of the inner sheet 100 may be produced by forming folds or bends at regular intervals along the length or width direction in a shape corresponding to a predetermined size and shape of the affected area as shown in FIG. 9 to form an uneven shape, or may be formed with folds or bends over part or the entirety of the inner sheet 100, or may be formed in a flat or gently curved shape. The supporting force of the polymer scaffold 10 for a prosthesis according to the present invention may be adjusted depending on such a shape of each inner sheet 100.

The present invention described above is not limited to the above-described embodiments and the accompanying drawings, and it will be apparent to one of ordinary skill in the art to which the present invention pertains that various substitutions, modifications and changes are possible without departing from the technical scope of the present invention.

[Brief Description of Main Parts]

```
TC: Taylor cone shape
  1: piston nozzle
  2: high-voltage electrode
  3: collector plate
  4: cooling dryer
  5: mold
  6: natural polymer solution
  7: natural polymer matrix
  8: synthetic polymer matrix
  9: fine nanofibers
  10: polymer scaffold for a prosthesis
according to the present invention
100: inner sheet
200: outer shell
```

1. A polymer scaffold for a prosthesis comprising:
an inner sheet formed into a certain size and shape and made of a biodegradable synthetic polymer material; and
an outer shell made of a biodegradable natural polymer material in the form of a sealed pouch surrounding the inner sheet;
wherein the inner sheet is formed by cutting a portion of a synthetic polymer matrix, which is made of the biodegradable synthetic polymer material and has a larger area and thickness than the inner sheet, into a predetermined shape and size, so that the synthetic polymer matrix has a network-like structure in which fine nanofibers are formed by amorphous stacking, which makes it possible to control the diameter and the stacking density of the fine nanofibers during a stacking process of the fine nanofibers, so that a material of the inner sheet can be determined depending on the diameter and the stacking density of the fine nanofibers constituting the inner sheet, and the overall shape and elasticity characteristics can be determined depending on the shape, size, and thickness of the inner sheet, or
the inner sheet is formed by 3D printing, and the inner sheet is formed with bends or folds at regular intervals along the length or width direction in a shape corresponding to a predetermined size and shape of the affected area to form an uneven shape, or is formed with bends or folds partially or entirely, or is formed in a flat or gently curved shape or a certain geometric shape, and the inner sheet is formed of one layer of one sheet-shaped member or formed by overlapping two or more layers of one sheet-shaped member.

2. The polymer scaffold of claim 1, wherein the synthetic polymer matrix is formed into a micro-network structure by amorphously stacking synthetic polymer fibers that are injected by applying a high voltage to a piston nozzle, and
a plurality of synthetic polymer matrices having different densities and porosities are provided by differently forming the thickness of the fibers and the stacking distance between the fibers depending on the viscosity of a synthetic polymer solution charged into the piston nozzle and the magnitude of the high voltage, so that the inner sheet is provided with a plurality of sheets having different densities and porosities.

3. The polymer scaffold of claim 1, wherein the material of the synthetic polymer matrix includes a component composed of any one or a combination of two or more selected from poly (L-lactic acid) (PLA), poly (glycolic acid) (PLGA), poly (ε-caprolactone) (PCL), and poly (L-lactide-co-ε-carprolactone (PLCL).

4. The polymer scaffold of claim 1, wherein the natural polymer material includes a component composed of any one or a combination of two or more selected from collagen type 1, sodium hyaluronate (HA), and chondroitin sulfate.

5. A method of manufacturing a polymer scaffold for a prosthesis, comprising:
manufacturing a mold in a shape corresponding to a predetermined shape of an affected area;
manufacturing an inner sheet by mixing a biodegradable synthetic polymer fuel;
inserting the inner sheet into the mold;
preparing a natural polymer solution by mixing a biodegradable natural polymer fuel;
injecting the natural polymer solution into the mold;
drying the natural polymer solution injected into the mold; and
removing the mold,
wherein the size and shape of the polymer scaffold can be adjusted to correspond to the affected area of the patient and treatment period by changing the size and shape of the mold according to the age of a patient, the size of the affected area, and the treatment period.

6. The method of claim 5, wherein the manufacturing of the inner sheet includes:
first manufacturing a synthetic polymer matrix having a certain thickness and shape by mixing the biodegradable synthetic polymer solution, and then cutting a portion of the synthetic polymer matrix to an area and thickness that are insertable into the mold to manufacture the inner sheet, or
manufacturing the inner sheet by injecting the biodegradable synthetic polymer fuel by 3D printing to mold the biodegradable synthetic polymer fuel into a specific shape.

7. The method of claim 6, wherein the manufacturing of the synthetic polymer matrix includes dispersing the synthetic polymer solution by spraying the synthetic polymer solution through a piston nozzle, wherein the synthetic polymer sprayed from the nozzle is formed in the form of fine fiber strands while weakening the surface tension of the synthetic polymer by disposing a high-voltage electrode at the nozzle and applying a high voltage to the nozzle, and the fiber form is produced by sequentially amorphously stacking the fiber strands on a predetermined collector plate having a certain area.

8. The method of claim 7, wherein the manufacturing of the synthetic polymer matrix includes adjusting the porosity and density of the completed synthetic polymer matrix by increasing or decreasing the viscosity of the synthetic polymer solution to reduce or increase the thickness of the fiber strands, increasing or decreasing the magnitude of the high voltage to reduce or increase the thickness of the fiber strands, and decreasing or increasing the inflow speed of the synthetic polymer solution introduced into the piston nozzle to reduce or increase the thickness of the fiber strands.

9. The method of claim 8, wherein the manufacturing of the synthetic polymer matrix includes further decreasing the diameter of the fiber strands spun from the piston nozzle by adding a salt to the synthetic polymer solution to increase the surface charge density of the synthetic polymer sprayed from the piston nozzle.

10. The method of claim 6, wherein the manufacturing of the inner sheet includes:
cutting a portion of the synthetic polymer matrix from the synthetic polymer matrix to an area and thickness that are insertable into the mold, and
forming an uneven shape by forming bends or curves on the cut portion of the synthetic polymer matrix at regular intervals along the length direction in a shape corresponding to a predetermined size and shape of the affected area, or forming the cut portion into a three-dimensional shape by performing bend or curve processing on a part or entirety of the cut portion.

11. The method of claim 6, wherein when the biodegradable synthetic polymer fuel is injected by 3D printing to mold the biodegradable synthetic polymer fuel into a specific shape,
the specific shape is produced by forming curves, protrusions, or perforations at regular intervals, forming the inner sheet to have a gentle curve over the entire area thereof, or molding the inner sheet to have a specific geometric shape, and
the inner sheet is formed of one sheet or formed by overlapping two or more sheets.
